(19) United States
(12) Patent Application Publication
(10) Pub. No.: US 2004/0204466 A1
(43) Pub. Date: Oct. 14, 2004

(54) Treatment of bronchial asthma using 5-HT3 receptor antagonists

(75) Inventors: Shiv Kumar Agarwal, Chennai (IN); Devendra Kumar Agrawal, Omaha, NE (US)

Correspondence Address: OLIFF & BERRIDGE, PLC, P.O. BOX 19928, ALEXANDRIA, VA, 22320, US

(73) Assignee: Orchid Chemicals and Pharmaceuticals Ltd., Chennai (IN)

(21) Appl. No.: 10/412,686

(22) Filed: Apr. 14, 2003

(51) Int. Cl. A61K 31/4178

(52) U.S. Cl. 514/381; 514/397

(57) ABSTRACT

Accordingly, the present invention provides a method of treating bronchial asthma in a subject, said method comprising the step of administering an effective amount of a 5-HT₃ receptor antagonists optionally in combination with one or more pharmaceutically acceptable excipients and more particularly the present invention relates to a method of treating bronchial asthma using ondansetron, its polymorphs, its solvates or its pharmaceutically acceptable salts.

### FIELD OF THE INVENTION

The present invention relates to a method of treating bronchial asthma using 5-HT₃ receptor antagonists. More particularly the present invention relates to a method of treating bronchial asthma using ondansetron, its polymorphs, its solvates or its pharmaceutically acceptable salts. The present invention also relates to a pharmaceutical composition containing ondansetron, its polymorphs, its solvates, its pharmaceutically acceptable salts for the treatment of bronchial asthma.

### BACKGROUND OF THE INVENTION

The 5-HT₃ receptor antagonists comprise a defined and recognized class of pharmaceutically active compounds well known in the art and characterized by their pharmacological activity. Various 5-HT₃ receptor antagonist compounds are commercially available and clinically applied, e.g. in the treatment of emesis.

Bronchial asthma is reversible airway obstruction. Asthma has become a modern epidemic in Western society. Currently the most common chronic childhood disease is asthma, which affects roughly 15 million people in the United States alone. A potential sole genetic etiology is very unlikely when the rapid increase in the incidence of asthma is taken into account. Allergic asthma is a disease of chronic inflammation marked by periods of airway hyperresponsiveness (AHR) to one or more innocuous environmental allergens. The hallmarks of asthma include pulmonary inflammation, AHR, bronchoalveolar lavage (BAL) and tissue eosinophilia, and TH₂ lymphocyte profile.

Histamine is a primary mediator responsible for the increases in nasal mucus secretion, rhinorrhea, sneezing and pruritis found in allergic rhinitis. Although antihistamines are the primary treatment for allergic rhinitis, recent findings indicate that the antiallergic efficacy of antihistamines against mast cell mediated nasal congestion may be expanded by co-treatment with a histamine H₃ receptor antagonist.

Histamine H₃ receptors are widely distributed on peripheral autonomic nerves, and are found presynaptically on postganglionic nerve terminals. Activation of these receptors inhibits neurally induced cholinergic contraction in guinea pig trachea, human bronchus, and guinea pig ileum. These receptors are also found presynaptically on postganglionic sympathetic nerves, and activation attenuates a variety of effector responses. H₃ receptors have also been reported to modulate vascular responses by inhibition of norepinephrine release from sympathetic nerve terminals.

Among the airway diseases bronchial asthma and COPD (chronic obstructive pulmonary disease) are most prevalent. COPD is a syndrome of disorders involving inflammation of large and small airways and lung parenchyma. COPD is an irreversible and the nature of infiltrate is different from bronchial asthma, which is reversible.

The following literature references show the different uses of histamine receptor antagonists:

WO 01/10423 describes new use, in particular a new pharmaceutical use for compounds having 5-HT₃ receptor antagonist activity, for a new treatment of pulmonary/bronchial obstructive diseases, especially (1) Chronic Obstructive Pulmonary Disease (COPD); (2) Adult Respiratory Distress Syndrome (ARDS); or, in a broader sense of the invention, also (3) bronchitis of whatever type or genesis including, e. g., chronic, acute, arachidic, catarrhal, croupous, chronic or phthinoid bronchitis; or (4) pneumoconiosis of whatever type or genesis, including, for example, aluminosis, anthracosis, asbestosis, ptilosis, siderosis, silicosis, tabacosis and byssinosis; (5) laryngospasm; (6) pulmonary vasculitis; (7) pulmonary granulomatosis; or (8) extrinsic allergic alveolitis; or any combination of more than one of the diseases mentioned above.

WO 00/64441 describes compound having agonist activity to the 5-HT₄ receptor for use as a medicament and to the use of said compound in the manufacture of a medicament for therapeutic or prophylactic treatment of disorders involving bronchoconstriction of a human or animal body, as well as methods of treatment.

The publication also describes compounds having antagonist activity to the 5-HT₂ₐ receptor for use as a medicament and to the use of said compound in the manufacture of a medicament for therapeutic or prophylactic treatment of disorders involving bronchoconstriction of a human or animal body, as well as methods of treatment.

Both asthma and COPD are identified by the presence of characteristic symptoms and functional abnormalities. The airway obstruction is the sine qua non of both COPD and asthma. The airway obstruction in asthma must be reversible to establish a diagnosis, where as COPD is identified as a syndrome characterized by abnormal tests of expiratory flow that do not change marketedly over periods of several months of observation (American Thoracic Society (1987): Standards for the diagnosis and care of patient with chronic obstructive pulmonary diseases (COPD) and asthma. Am. Rev. Respir. Dis 136:225-244. Thus, COPD is characterized by irreversible airway obstruction, whereas asthma is reversible.

### OBJECTS OF THE INVENTION

The main object of the present invention is to provide a method of treating bronchial asthma in a subject using 5-HT₃ receptor antagonists.

Another object of the present invention is to provide a method for treating bronchial asthma in mammals including human beings using ondansetron, its polymorphs, its solvates or its pharmaceutically acceptable salts.

### BRIEF DESCRIPTION OF THE ACCOMPANYING DRAWINGS

In the drawings accompanying the specification,

FIG. 1 represents the effect of ondansetron treatment on early allergic response to antigen challenges in ovalbumin sensitized and challenged mice.

FIG. 2 represents the effect of ondansetron treatment on late allergic response to antigen challenges in ovalbumin sensitized and challenged mice.

FIG. 3 represents the effect of dexamethasone on early allergic response to antigen challenges in ovalbumin sensitized and challenged mice.

FIG. 4 represents the effect of dexamethasone on late allergic response to antigen challenges in ovalbumin sensitized and challenged mice.

FIG. 5 represents the effect of Ondansetron on airway hyperresponsiveness to methacholine in ovalbumin sensitized and challenged mice.

FIG. 6 represents the PC 200 values for methacholine pre and post antigen challenge in ovalbumin sensitized and challenged mice.

FIG. 7 represents the effect of Ondansetron on total number of cells in the BAL fluid of ovalbumin-sensitized and challenged mice.

FIG. 8 represents the effect of Ondansetron on percent Eosinophils in the BAL fluid of ovalbumin-sensitized and challenged mice.

### DETAILED DESCRIPTION OF THE PRESENT INVENTION

Accordingly, the present invention provides a method of treating bronchial asthma in a subject, said method comprising the step of administering an effective amount of a 5-HT₃ receptor antagonists optionally in combination with one or more pharmaceutically acceptable excipients.

In an embodiment of the present invention, the term “treating” includes therapeutic and prophylactic treatment of bronchial asthma.

In another embodiment of the present invention, the term “treating” includes alleviation, amelioration or control of the diseases, processes, conditions or events.

In yet another embodiment of the present invention, bronchial asthma is of reversible type.

In still another embodiment of the present invention, the 5-HT₃ receptor antagonists prevent antigen-induced airway resistance of the late allergic response (LAR), airway hyperresponsiveness (AHR).

In one more embodiment of the present invention, the 5-HT₃ receptor antagonists prevent ovalbumin-induced airway resistance of the late allergic response (LAR), airway hyperresponsiveness (AHR).

In one another embodiment of the present invention, the subject is a mammal including human being.

In a further embodiment of the present invention, the 5-HT₃ receptor antagonists are selected from the group consisting of Ondansetron, Granisetron, Dolasetron, Tropisetron, Ramosetron, Fabesetron, Lintopride, Alosetron, their polymorphs, solvates and its pharmaceutically acceptable salts.

In another embodiment of the present invention, the 5-HT₃ receptor antagonist is Ondansetron or its polymorphs or its solvates or its pharmaceutically acceptable salts.

In yet another embodiment of the present invention, the 5-HT₃ receptor antagonists are administered in the form of capsules, tablets, parenterals, aerosols, syrups, solutions, suspensions, emulsions, suppositories and topical applications such as gels, ointments, transdermal patches.

In still another embodiment of the present invention, the pharmaceutically acceptable excipients includes diluents, disintegrents, lubricants, glidents, plasticizers, solubilizing agents, wetting agents, buffering agents, surfactants, absorption enhancers, preservatives, sweeteners and binding agents.

In one more embodiment of the present invention, 10 to 150 μg/kg of the 5-HT₃ receptor antagonists optionally in combination with one or more pharmaceutically acceptable excipients is administered.

In one another embodiment of the present invention, 50 to 150 μg/kg of the 5-HT₃ receptor antagonists in combination with one or more pharmaceutically acceptable excipients is administered.

In a further embodiment of the present invention, the 5-HT₃ receptor antagonists is administered once, twice or thrice a day.

The present invention is based on the novel finding that certain 5-HT₃ receptors prevents antigen-induced (in this case ovalbumin) airway resistance of the late allergic response (LAR), airway hyperresponsiveness (AHR) without effecting the early allergic response (EAR) and therefore suitable as agents for treatment of bronchial asthma. Accordingly, the present invention, relates to the use of 5-HT₃ receptor antagonists for the treatment of bronchial asthma.

The invention more particularly relates to the use of 5-HT₃ receptor antagonists as a medicament, wherein 5-HT₃ receptor antagonist prevents antigen-induced airway resistance of the LAR, AHR without affecting IgE.

The present invention also provides a pharmaceutical composition containing an effective amount of 5-HT₃ receptor antagonist and other pharmaceutically acceptable excipients.

The pharmaceutical compositions of the present invention are in conventional forms, for example capsules, tablets, aerosols, syrups, solutions, suspensions, topical applications and the like, may contain flavourants, sweeteners etc., in suitable solid or liquid carriers or diluents

For nasal administration, the preparation may contain ondansetron dissolved or suspended in a liquid carrier, in particular an aqueous carrier, for aerosol application. The carrier may contain additives such as solubilizing agents, e.g., propylene glycol, surfactants, absorption enhancers such as lecithin (phosphatidylcholine) or cyclodextrin, or preservatives such as parabens.

The present invention further described in detail in the following examples which are given by way of illustration and hence, should not be construed to limit the scope of the present invention in any manner.

The activity of 5-HT₃ receptor antagonist of the present invention for the treatment of bronchial asthma has been found by the following methodology.

### EXAMPLE 1

Four to five-week old female Balb/c mice (Harlan Laboratories, Indianapolis, Ind.) were housed according to the NIH guidelines and were allowed constant access to food and water. Intraperitoneal injections of 6 μg of grade V chicken egg ovalbumin (Ova; Sigma-Aldrich, St. Louis, Mo.) adsorbed to 2.25 mg Imject Alum (Pierce, Rockford, Ill.) and dissolved in PBS. Aerosol sensitization with 0.2% ovalbumin (Ova) for 10 minutes using Ultra-Neb 90 nebulizer (DeVilbiss, Somerset, Pa.).

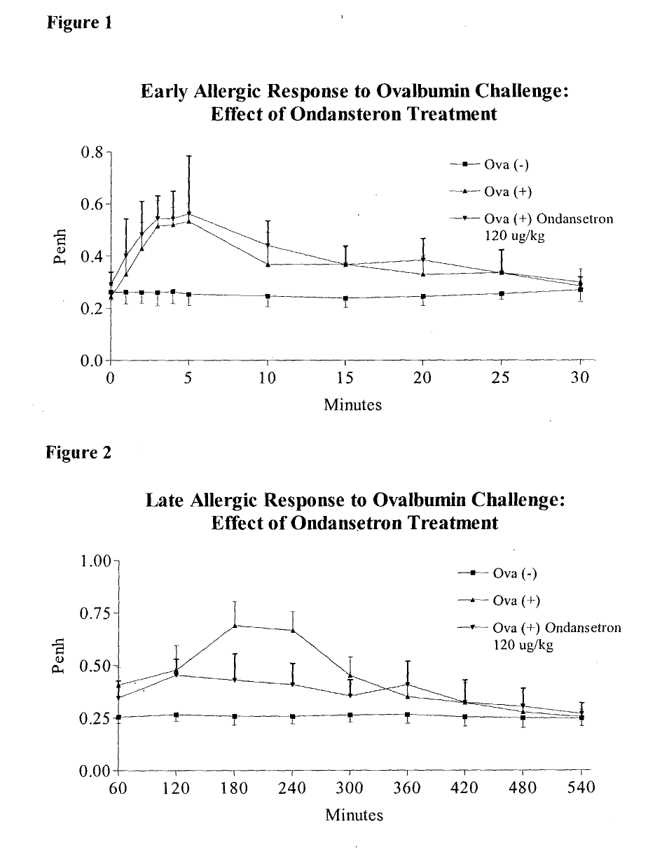

Ondansetron was administered by gavage in the dose of 120 μg/kg twice a day for 10 days. Analysis of Ova-specific airway resistance of both the EAR and LAR revealed that nonsensitized control mice had significantly lower resistance during either phase (p <0.05 for the EAR and p <0.001 for the LAR compared to sensitized controls). Ondansetron-treated mice exhibited significant protection during the LAR (FIG. 2) (P <0.001) but was without EAR protection (FIG. 1).

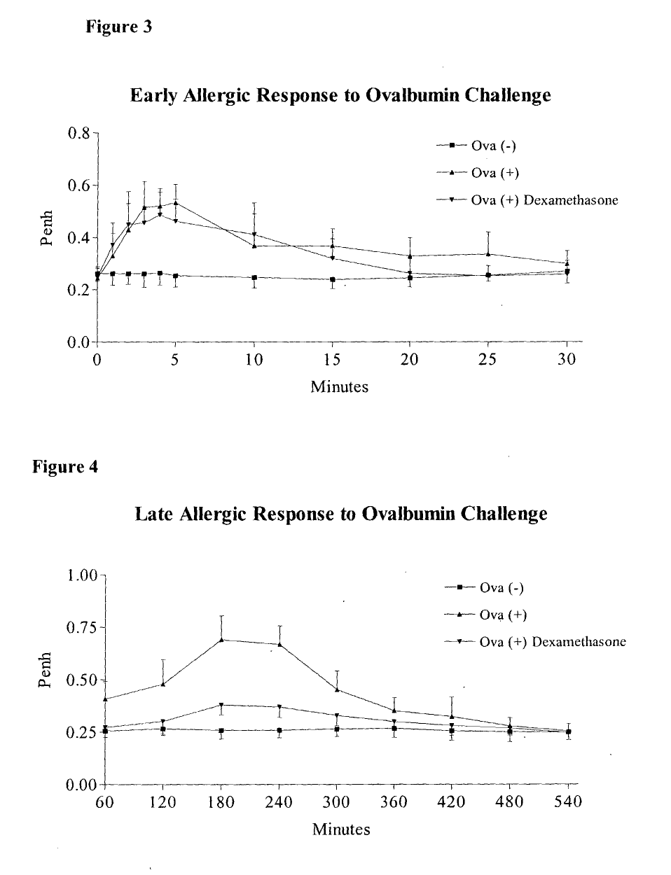

This finding, that the EAR was unaffected, was further corroborated by the analysis of serum IgE levels. Not only were mean serum IgE concentrations unaffected by ondansteron treatment, the values were increased, albeit insignificantly. The results, which examined B cell (antigen presenting cell)/T cell interactions during sensitization were unaffected by ondansteron administration, as IgE levels were unchanged. This effect was comparable to dexamethasone (FIGS. 3 and 4).

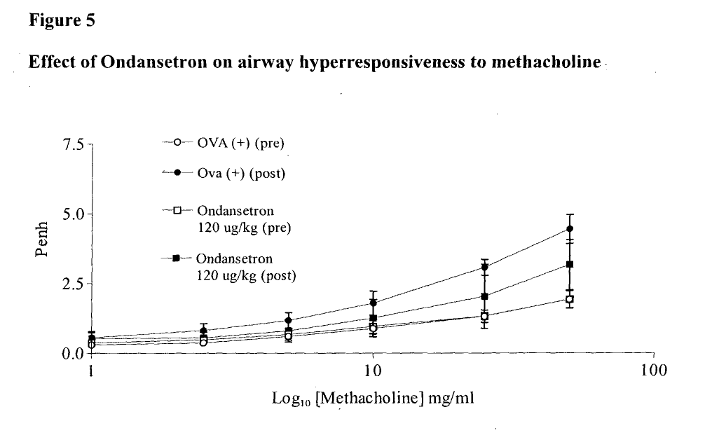

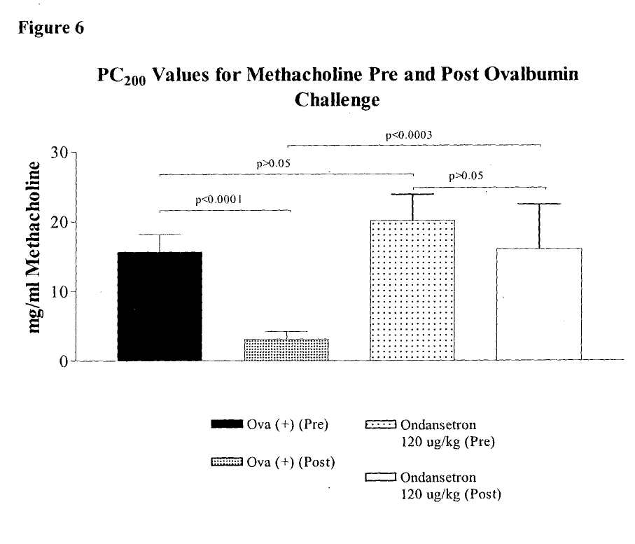

Twenty-four hours after antigen challenge, the mice were challenged with increasing does of methacholine and Penh values were recorded by barometric plethysmography. In support of the suppressed LAR, AHR to methacholine was also completely suppressed after ondansetron treatment (FIGS. 5 and 6).

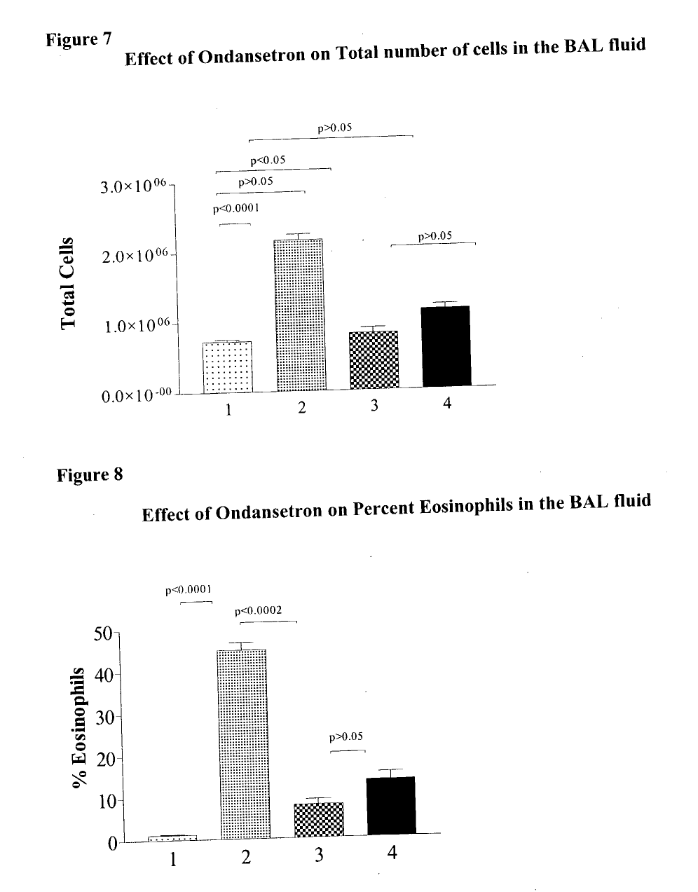

Although not universally accepted the LAR as well as AHR have been positively linked to the presence of eosinophils in the airways. Interleukin-5 is a critical growth factor for eosinophil and act as a specific group factor and potent stimulant and chemotactic factor. Indeed, the Inventors observed significantly descried numbers of eosinophil in BAL immediately following methacholine challenge (FIGS. 7 and 8).

Direct ondansetron activity on the challenge is unlikely as the last injection of ondansetron was given 3 days previously. In addition, the Inventors do not except that direct effect of the earlier ondansetron exposure is responsible for the decrease in asthmatic symptoms and is likely to occur via increased its effect on postganglionic and presynaptic cholinergic nerve terminals.

We claim:

1. A method of treating bronchial asthma in a subject, said method comprising the step of administering an effective amount of a 5-HT₃ receptor antagonists optionally in combination with one or more pharmaceutically acceptable excipients.

2. A method as claimed in claim 1, wherein “treating” includes therapeutic and prophylactic treatment of bronchial asthma.

3. A method as claimed in claim 1, wherein “treating” includes alleviation, amelioration or control of the diseases, processes, conditions or events.

4. A method as claimed in claim 1, wherein the wherein bronchial asthma is of reversible type.

5. A method as claimed in claim 1, wherein the 5-HT₃ receptor antagonists prevent antigen-induced airway resistance of the late allergic response (LAR), airway hyperresponsiveness (AHR).

6. A method as claimed in claim 5, wherein the 5-HT₃ receptor antagonists prevent ovalbumin-induced airway resistance of the late allergic response (LAR), airway hyperresponsiveness (AHR).

7. A method as claimed in claim 1, wherein the subject is a mammal including human being.

8. A method as claimed in claim 1, wherein the 5-HT₃ receptor antagonists are selected from the group consisting of Ondansetron, Granisetron, Dolasetron, Tropisetron, Ramosetron, Fabesetron, Lintopride, Alosetron, their polymorphs, solvates and its pharmaceutically acceptable salts.

9. A method as claimed in claim 8, wherein the 5-HT₃ receptor antagonist is Ondansetron or its polymorphs or its solvates or its pharmaceutically acceptable salts.

10. A method as claimed in claim 1, wherein the 5-HT₃ receptor antagonists are administered in the form of capsules, tablets, parenterals, aerosols, syrups, solutions, suspensions, emulsions, suppositories and topical applications such as gels, ointments, transdermal patches.

11. A method as claimed in claim 1, wherein the pharmaceutically acceptable excipients includes diluents, disintegrents, lubricants, glidents, plasticizers, solubilizing agents, wetting agents, buffering agents, surfactants, absorption enhancers, preservatives, sweeteners and binding agents.
